(19) United States
(12) Patent Application Publication
(10) Pub. No.: US 2024/0375100 A1
(43) Pub. Date: Nov. 14, 2024

(54) MICROFLUIDIC SYSTEM AND METHOD OF USE AND MEASUREMENT

(71) Applicant: Purdue Research Foundation, West Lafayette, IN (US)

(72) Inventors: Hui Ma, West Lafayette, IN (US); Tamara L. Kinzer-Ursem, West Lafayette, IN (US); Jacqueline C. Linnes, West Lafayette, IN (US)

(73) Assignee: Purdue Research Foundation, West Lafayette, IN (US)

(21) Appl. No.: 18/657,406

(22) Filed: May 7, 2024

Related U.S. Application Data
(60) Provisional application No. 63/465,103, filed on May 9, 2023.

(51) Int. Cl. B01L 3/00

(52) U.S. Cl. CPC B01L 3/502715; B01L 2200/16; B01L 2300/0663; B01L 2300/0816; B01L 2300/0867; B01L 2300/0883; B01L 2400/0487

(57) ABSTRACT

A microfluidic device is disclosed which includes two or more solution inlets each adapted to be selectively opened and closed and each adapted to receive a respective solution, two or more loading channels each coupled to a respective solution inlet and each adapted to hold the respective solution, a fluidic force inlet coupled to the two or more solution inlets and adapted to provide a fluidic force to each respective loading channel, and a mixing channel coupled to the two or more loading channels and adapted to mix each solution held in each of the two or more loading channels when a fluidic force is applied, the mixing channel terminating at a trap zone adapted to receive the mixed solution from the mixing channel, the trap zone including a fluidic force outlet adapted to release the received fluidic force.

### CROSS-REFERENCE TO RELATED APPLICATIONS

The present non-provisional patent application is related to and claims the priority benefit of U.S. Provisional Patent Application Ser. 63/465,103, filed May 9, 2023, the contents of which are hereby incorporated by reference in its entirety into the present disclosure.

### STATEMENT REGARDING GOVERNMENT FUNDING

This invention was made with government support under AI140474 awarded by the National Institutes of Health. The government has certain rights in the invention.

### TECHNICAL FIELD

The present disclosure generally relates to microfluid systems and in particular to a microfluidic system for protein-protein interaction and method of interaction measurement utilizing particle diffusometry.

### BACKGROUND

This section introduces aspects that may help facilitate a better understanding of the disclosure. Accordingly, these statements are to be read in this light and are not to be understood as admissions about what is or is not prior art.

Protein-protein interactions play a role in almost all biological processes of living organisms. Accurate measurement and characterization of their binding kinetics are needed in both in vivo and in vitro biological and biomedical research. Conventional gold standard assays of protein-protein interaction measurement methods such as surface plasma resonance (SPR), quartz crystal microbalance (QCM) and bio-layer interferometry (BLI) can generate real-time and precise data. However, those methods are costly to perform due to the high cost of instruments, consumables, and reagents.

Continuous flow and droplet microfluidic devices with rapid mixing have been used to analyze biomolecular interactions in experiments that are otherwise prohibitively difficult to implement. Microfluidic methods provide highly controlled fluid flow at low Reynolds numbers and are often considered low-cost because an extremely low sample volume is sufficient to perform an analysis. However, many researchers only count the volume of the sample that is immediately analyzed, typically the volume of the microchannel, sample droplet, or other working zones alone rather than the total experimental volume. The actual amount of sample used can be much more than claimed, as the experimental setup often requires syringes and tubing filled with solution upstream of the reaction zone. Even for the two-phase droplet-based microfluidics using tiny droplet volume (in the picoliter range), usually much more sample solution (˜mL) than the single droplet volume is needed for the experiments.

Furthermore, a common problem for both continuous flow and droplet-based two-phase flow is that stabilization is required before analysis. The syringe pump must be run prior to analysis to remove trapped bubbles and allow the flow to develop and stabilize fully in continuous flow experiments. For droplet-based flow, consistent and uniform droplets can only be formed after the system stabilizes. This initial unsteady period wastes the sample solution. Additionally, biomolecular reagents are usually very expensive or hard to collect and purify, and thus the excess fluid volume requirement increases the experimental burden and cost significantly. With these problems, existing methods and microfluidic chips cannot fully leverage the low-volume and low-cost advantages of microfluidics.

Therefore, there is an unmet need for a novel microfluidic system that can overcome excess solution requirements of the prior art.

### SUMMARY

A microfluidic device is disclosed. The microfluidic device includes two or more solution inlets each adapted to receive a respective solution, each of the two or more solution inlets adapted to be selectively opened and closed, two or more loading channels each coupled to a respective inlet of the two or more solution inlets, each loading channel adapted to hold the respective solution, a fluidic force inlet coupled to the two or more solution inlets and adapted to provide a fluidic force to each respective loading channel, and a mixing channel coupled to the two or more loading channels, thereby adapted to mix each solution held in each of the two or more loading channels when a fluidic force is applied to the fluidic force inlet. The mixing channel terminates at a trap zone adapted to receive the mixed solution from the mixing channel when exposed to fluidic force, the trap zone includes a fluidic force outlet adapted to release the received fluidic force.

A microfluidic system is also disclosed. The microfluidic system includes two or more solution inlets each adapted to receive a respective solution, each of the two or more solution inlets adapted to be selectively opened and closed, two or more loading channels each coupled to a respective inlet of the two or more solution inlets, each loading channel adapted to hold the respective solution, a fluidic force inlet coupled to the two or more solution inlets and adapted to provide a fluidic force to each respective loading channel, and a mixing channel coupled to the two or more loading channels, thereby adapted to mix each solution held in each of the two or more loading channels when a fluidic force is applied to the fluidic force inlet. The mixing channel terminates at a trap zone adapted to receive the mixed solution from the mixing channel when exposed to fluidic force, the trap zone includes a fluidic force outlet adapted to release the received fluidic force. The microfluidic system also includes a microscope system optically coupled to the trap zone adapted to irradiate the mixed solution with a light source and return optical emission from the mixed solution to an image capture device.

### BRIEF DESCRIPTION OF FIGURES

FIG. 1 is a schematic of a microfluidic device, according to the present disclosure.

FIG. 2A is a schematic of a microfluidic system incorporating the microfluidic device of FIG. 1, according to the present disclosure.

FIG. 2B is a schematic of the microfluid device of FIG. 1, with a fluidic force device (according to one embodiment, a syringe) in an un-activated position coupled to a fluidic force inlet.

FIG. 2C is the schematic of FIG. 2B with a first fluid dispensing device (according to one embodiment, a pipette) coupled to a first solution inlet.

FIG. 2D is the schematic of FIG. 2C with a first fluid in the first fluid dispensing device transferred to a first loading channel.

FIG. 2E is the schematic of FIG. 2D with a second fluid dispensing device (according to one embodiment, a pipette) coupled to a second solution inlet.

FIG. 2F is the schematic of FIG. 2E with a second fluid in the second fluid dispensing device transferred to a second loading channel.

FIG. 2G is the schematic of FIG. 2F, with the first and second solution inlets each sealed by an inlet closing device (according to one embodiment, a piece of pressure sensitive adhesive tape).

FIG. 2H is the schematic of FIG. 2G with the fluidic force device shown in an intermediate activation status, thus forcing the first and second fluids to the mixing channel, thereby mixing the first and second fluids.

FIG. 2I is the schematic of FIG. 2H with the fluidic force device shown in a final activation status, thereby forcing the mixed first and second fluids to a trap zone.

FIG. 2J is a close-up schematic depicting fluid (the first or second fluid) in the first or second fluid dispensing device positioned in the first or second solution inlet and ready to release the first or second fluid into the first or second loading channel.

FIG. 2K is a schematic and a microscope system measuring fluid in the trap zone of the microfluid device of the present disclosure.

FIGS. 3A, 3B, and 3C depict two particle diffusometry images with one or more segments across two timeslots with representations of particles in one or more segments (FIG. 3A); showing diffusivity of particles across time (FIG. 3B); and autocorrelation of an interrogation window with itself producing a high and narrow auto-correlation peak and cross-correlation of an interrogation window with a corresponding interrogation window in the next image in the stack produces a low and wide cross-correlation peak (FIG. 3C).

FIG. 4 is a schematic of another microfluidic system incorporating a second embodiment of the microfluidic device similar to FIG. 1, according to the present disclosure.

FIG. 5 is a schematic of yet another microfluidic system incorporating a third embodiment of the microfluidic device similar to FIG. 1, according to the present disclosure.

FIG. 6 is a flowchart showing steps performed by a processor, according to the present disclosure.

### DETAILED DESCRIPTION

For the purposes of promoting an understanding of the principles in the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

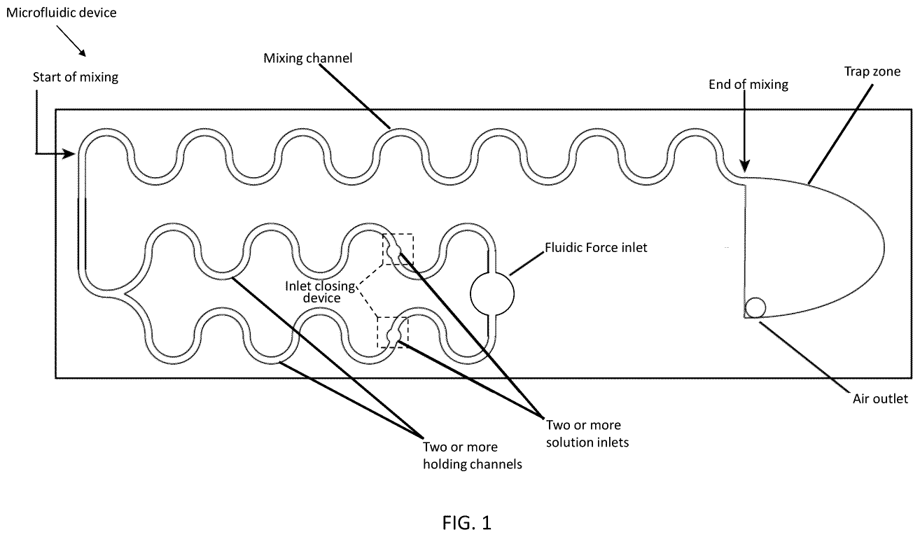

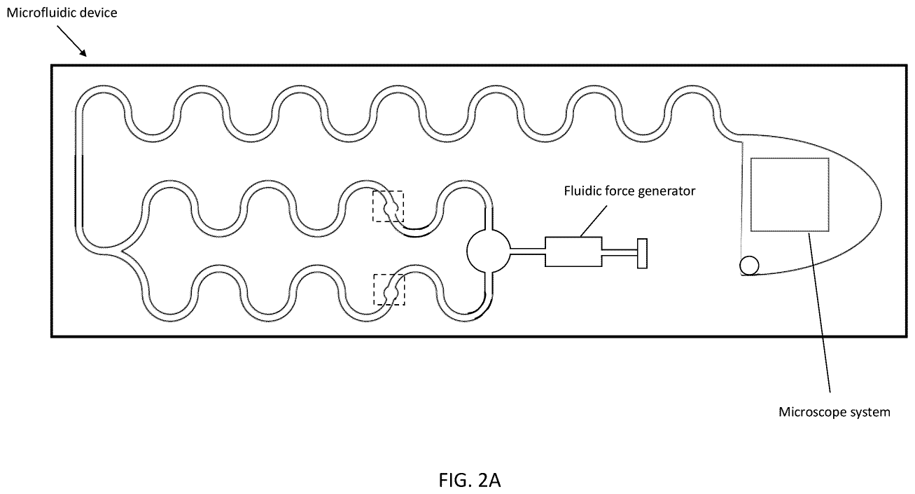

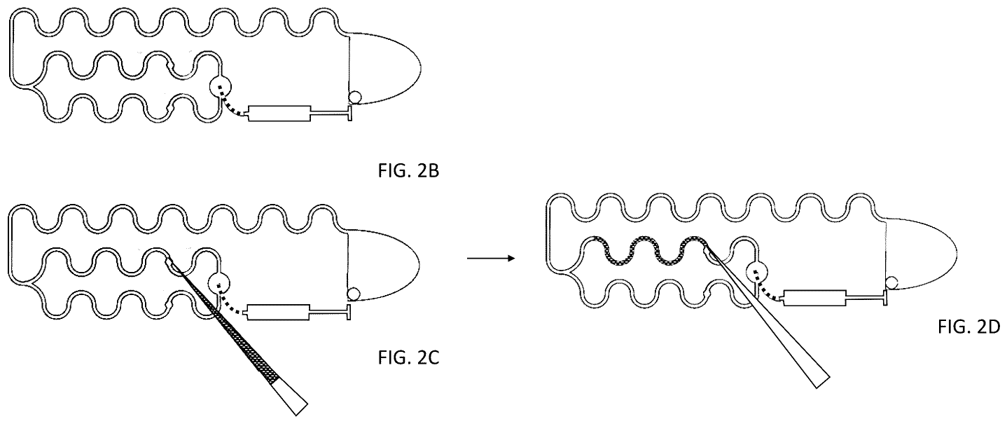

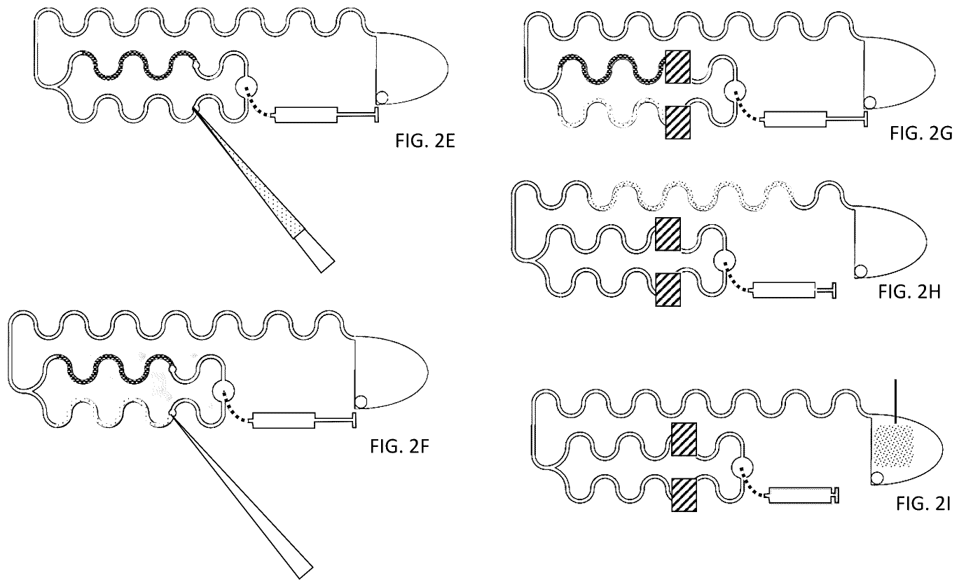

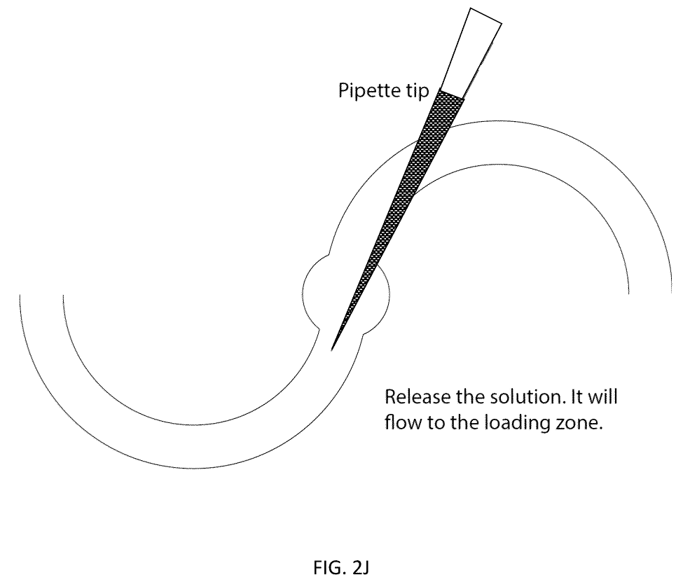

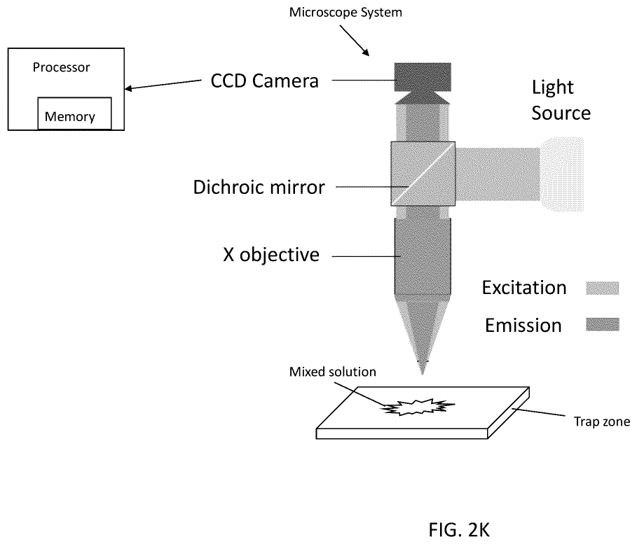

A novel microfluidic system is disclosed herein that can overcome excess solution requirements of the prior art as well as a method of analyzing interactions between solutions introduced into the microfluidic system utilizing particle diffusometry. Towards this end, reference is made to FIGS. 1 and 2A are schematics of a first embodiment of a microfluid device (FIG. 1) and a part of a microfluidic system (FIG. 2A), according to the present disclosure. The microfluidic device includes two or more (e.g., a first and second) solution inlets each adapted to receive two or more (e.g., a first and second) fluids, respectively. Additionally, the microfluidic device includes two or more (e.g., a first and second) loading channels each coupled to a respective first and second solution inlets, and each of the first and second loading channels adapted to hold the first and second fluids, respectively. The fluidic device also includes a fluidic force inlet coupled to the first and second solution inlets and adapted to provide a fluidic force to each of the first and second loading channels, respectively. The microfluidic device further includes a mixing channel coupled to the first and second loading channels, thereby adapted to mix the first and second fluids held therein when a fluidic force is applied to the fluidic force inlet. The mixing channel terminates at a trap zone adapted to receive the mixed solution from the mixing channel when exposed to the fluidic force. The trap zone incudes a fluidic force outlet adapted to release the received fluidic force.

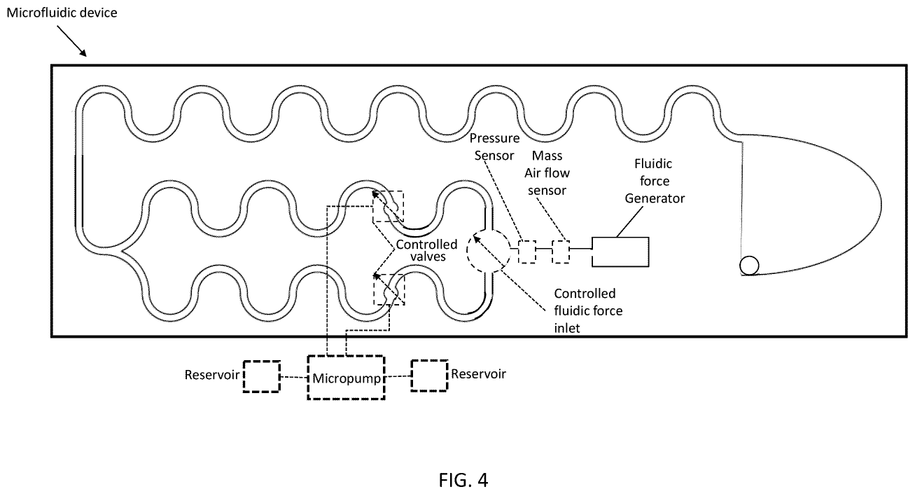

The microfluid system shown in FIG. 2A also includes a fluidic force generator (according to one embodiment, a syringe) coupled to the fluidic force inlet and is adapted to generate the fluid force discussed above. Additionally, the microfluidic system of FIG. 2A, included a microscope system. The microscope system is optically coupled to the trap zone and is adapted to irradiate the mixed solution with a light source and return optical emission from the mixed solution to an image capture device. In the microfluidic device, one or both of the first and second loading channels may be serpentine-shaped, thus adapted to hold a predetermined amount of the first or second fluid, respectively. In the microfluidic device, the mixing channel may be serpentine shaped, thus adapted to provide sufficient volume for mixing of the first and second fluids, based on a predetermined volume criterion. The mixing channel is defined by a length of between about 1 cm and about 20 cm and a diameter of about 1 μm and about 1 mm. In the microfluidic device, each of the first and second solution inlets may be adapted to be selectively closed and opened based on application of an inlet closing device (according to one embodiment, pressure sensitive adhesives). In the microfluidic device, the fluidic force inlet may be adapted to receive the fluid force from a syringe, or as described with respect to other embodiments depicted in FIG. 4, discussed below, a pump, a regulated compressed gas canister, or any other compressed gas source, accompanied with an optional mass air flow sensor, see FIG. 4. In the microfluidic device, each of the first and second solution inlets may include a one-way valve adapted to direct flow in a predefined direction.

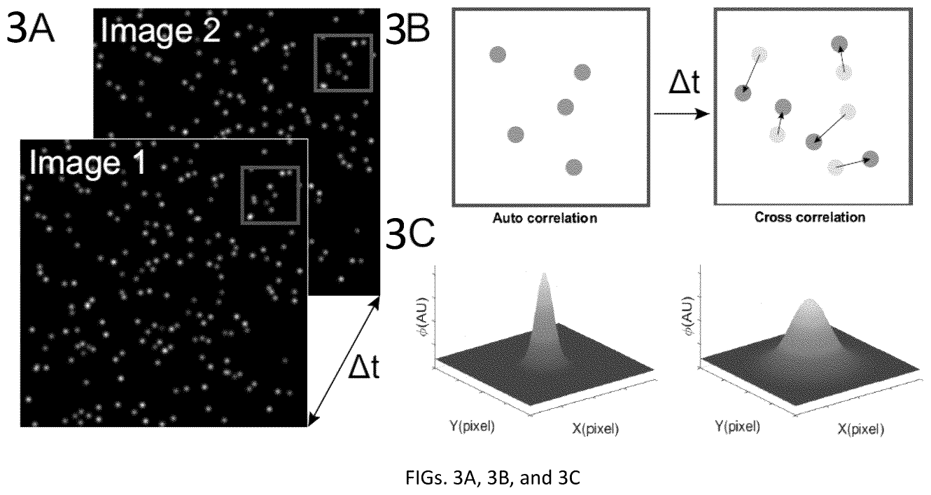

The microfluidic system may also incorporate a processor coupled to onboard or off-board non-transient memory housing instructions that are executed by the processor to obtain images from the trap zone, received from the image capture device, of the mixed solution. The processor in the microfluidic system may further be adapted to determine size of particles in the solution in the trap zone by application of particle diffusometry. Reference is made to U.S. Pat. No. 10,794,808 to Clayton et al., incorporated by reference in its entirety into the present disclosure, for an in-depth discussion of particle diffusometry. While the '808 patent provides a much more in-depth discussion of particle diffusometry, a brief description is provided herein. The processor may be programmed to divide a field of view of images captured by the image capture device into one or more segments. Reference is made to FIGS. 3A, 3B, and 3C which depict the one or more segments across two timeslots, representations of particles in the one or more segments, and autocorrelation and cross-correlation of particles for the first timeslot and across time from the first to the second timeslot. For each of the one or more segments, the processor may identify particles at two or more time intervals. For each of the two or more time intervals, the processor may obtain autocorrelation of first plurality of particles for a first timeslot according to locations of said first plurality of particles at the first timeslot. The processor then identifies an autocorrelation peak in the autocorrelation. Next, the processor obtains cross-correlation of second plurality of particles for a second timeslot according to locations of said second plurality of particles as compared to said first plurality of particles from the first timeslot. Next, the processor identifies a cross-correlation peak in the cross-correlation. Next, for each identified autocorrelation and cross-correlation peaks, the processor determines peak widths at a predetermined height, e.g., 1/e, of each peak. Next, the processor determines time-dependent particle size based on the determined peak widths as a function of time. According to one embodiment, the time-dependent particle size is determined based on:

$${d}_{p} = \frac{16 {k}_{B} T Δ t {M}^{2}}{3 π ( {s}_{o , c}^{2} - {s}_{o , a}^{2} )} ,$$

wherein dₚ is time-dependent diameter of particles,
k_{B} is the Boltzmann constant,
Tis temperature of the mixed solution,
μ is the mixed solution viscosity,
M is the magnification of the microscope of the microscope system, and
s_{o,c}² and s_{o,a}² are peak widths of peaks of the cross-correlation and autocorrelation, respectively.

The processor may also be programmed to determine time-dependent binding performance expressed as k_{obs}t between particles suspended in the respective solutions of the two or more loading channels. k_{obs}t is expressed as:

$${k}_{obst} t = log ( \frac{{V}_{m} - V ( t )}{{V}_{m} - {V}_{0}} ) ,$$

wherein V₀ represents initial volume of particles in one of the respective solutions of the two or more loading channels, and
V(t) represents time-dependent volume of the particles in the mixed solution based on the time-dependent particle size. Volume of a sphere can be determined based on 4/3πr³, where r is the radius of the sphere.

With the time-dependent volume of particles in the mixed solution known, k_{obs} at different times can be established based on the above equation. Once k_{obs} is known, a linear relationship between kₒₙ and k_{off} based on the concentration of particles in one or more of the solutions in the two or more loading channels can be established. These parameters define the binding performance of particles in the solutions of the two or more loading channels. In other words, using a linear equation:

$${k}_{obs} = {k}_{on} [ P 1 ] + {k}_{off} ,$$

wherein P1 represents concentration of particles in the solution in one of the two or more loading channels wherein

$$[ P 2 ] + [ P 1 ] {{⟶}^{kon}}_{{⟵}_{koff}} [ C ] ,$$

where [P2] represents concentration of particles in the solution in another of the two or more loading channels, and [C] represents the concentration of the particle complex (i.e., particles bound to each other) in the mixed solution in the trap zone.

Additional disclosure is found in Appendix-A and Appendix-B, filed herewith, contents of each of which is incorporated by reference into the present disclosure in its entirety.

Various parameters in connection with the microfluidic device shown in FIG. 1 is of particular importance. These include the channel width of the mixing channel (channel width can be between about 1 μm to about 1 mm), serpentine radius of the mixing channel (the radius can be between about 1 μm to about 5 cm), the channel length (the length can be between about 1 cm to about 20 cm), and an average velocity of mixed solution moving through the mixing channel (the average velocity can be between about 0.1 m/s to about 10 m/s). Time spent in the mixing channel is defined by 11u where l is the length of the mixing channel and u is the velocity of mixed solution traveling in the mixing channel. The radius of the mixing channel is governed by:

$$D e = {( \frac{L}{2 R} )}^{1 / 2} Re ,$$

where
De is the Dean number,
L is hydrodynamic diameter of the channel,
R is radius of serpentine path, and
Re is the Reynolds number defined by:

$$R e = \frac{ρ u L}{μ} ,$$

where p is density of the mixed solution,
μ is the viscosity of the mixed solution, and
u is the velocity of the mixed solution.

The mixer quality is characterized by the equation below.

$$E = \frac{{V}_{out}}{{V}_{in}} \\ {V}_{out} = \frac{1}{{S}_{out}} {\int }_{{s}_{out}} {( c - \frac{{c}_{0}}{2} )}^{2} dS \\ {V}_{in} = \frac{1}{{S}_{in}} {\int }_{{s}_{in}} {( c - \frac{{c}_{0}}{2} )}^{2} dS$$

where c is the concentration distribution of the species of interest, co is the initial concentration before mixing, Sₒᵤₜ and Sᵢₙ are the surface area of the outlet and inlet surfaces. If the mixer achieves ideal mixing, E=0; if the mixer does not mix solutions at all, E=1. Therefore, the better the mixer quality is, the smaller E is.

It should also be appreciated that the method disclosed herein is not limited to protein-protein interaction. The methodology can also be applied to other non-protein interactions, such as small molecule-protein biotin-streptavidin also works with the methods disclosed herein, which is demonstrated in Appendix-A. Other biomolecular binding is also within the scope of the present disclosure e.g. nucleic acid aptamer+small molecule, nucleic acid hybridization events, etc.

Referring to FIG. 4, a second embodiment of a microfluidic system is shown. The microfluidic system of FIG. 4 may further include a pressure sensor coupled to a controlled fluidic force inlet, the pressure sensor is adapted to generate a signal associated with pressure in first and second loading channels and the mixing channel of the microfluidic device. The microfluidic system may also include a temperature sensor in the trap zone adapted to provide a signal associated with temperature of the mixed solution in the trap zone. The two or more solution inlets shown in FIG. 1 are now replaced with controlled valves coupled to a micropump (e.g., a peristaltic pump) coupled to a respective reservoir holding a respective solution.

The microfluidic system of FIG. 4 may also include a processor coupled to the pressure sensor, the mass air flow sensor, the controlled fluidic inlet, the fluidic force generator, the temperature sensor, and a micropump coupled to a reservoir. The processor may include onboard or off-chip non-transient memory housing instructions that when executed allow the processor to receive pressure sensor signal, interpret the pressure sensor signal, receive mass air flow sensor, interpret the mass air flow sensor data, operate the fluid force generator, operate the controlled fluid force inlet, all in an open-loop control fashion according to a predetermined schedule, or in a closed-loop fashion based on the signal from the pressure sensor and the mass air flow as the feedback signals. Additionally, the processor may also receive temperature sensor signal and interpret the temperature sensor signal and use the temperature data in determining time-dependent particle size as discussed above.

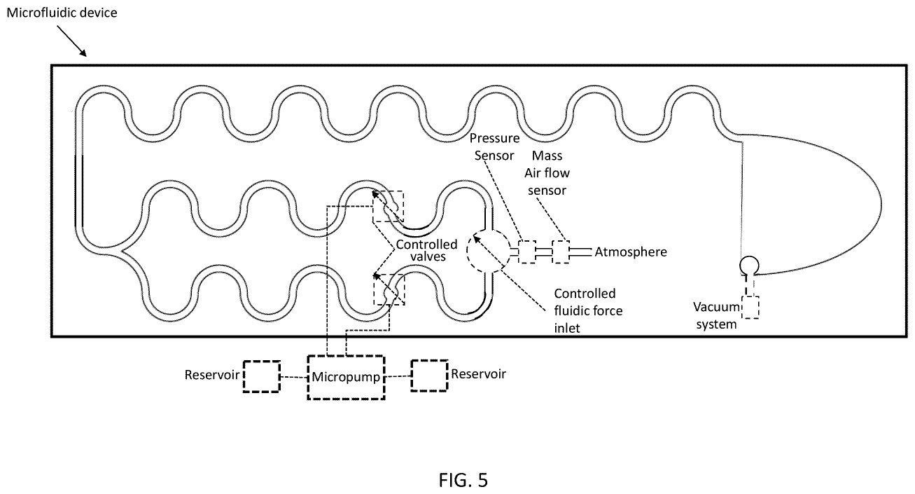

Referring to FIG. 5, an alternative embodiment is shown where the source of high pressure gas shown in FIG. 4 has been removed and replaced with a vacuum system coupled to the air outlet. The processor, described in relationship to FIG. 4 may be configured to control the vacuum system which causes air to be entered from the atmosphere into the microfluidic system. It should be noted that in FIGS. 4 and 5, the microscope system is not shown for simplicity, however, the microscope system is part of the microfluidic systems shown in these figure.

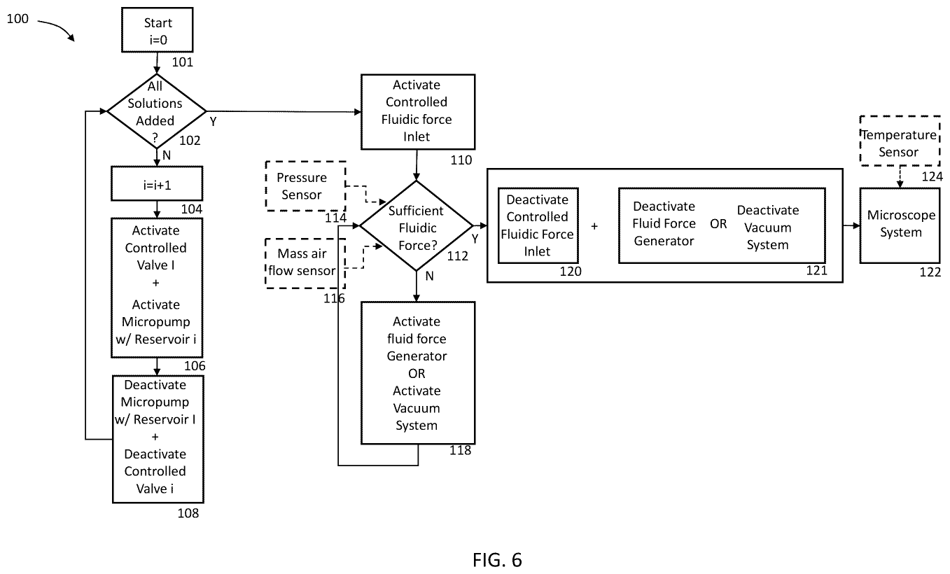

In operation, with reference to FIG. 6 a flowchart 100 describing operation of the system is provided as steps executed by the processor including receiving and interpreting optional receiving and interpreting pressure sensor data, receiving and interpreting optional air mass flow, control of fluid force generator, control of the controlled fluidic force inlet, control of the controlled valves each coupled to a respective solution inlet, and control of the micropump coupled to the reservoirs. The flowchart 100 starts at block 101 by initializing (e.g., setting a counter to 0), as provided by block 101. Next, the processor inquires as to whether all solutions have been added, as indicated by block 102. As indicated above, more than two solutions can be mixed by having at least two solution inlets each coupled to a respective loading channel. For example, at the initial state, neither of the solutions have been introduced. In step 102, if the counter has not reached the maximum count (i.e., answer “N”), the process continues to block 104 where the counter is incremented by one. Next, the processor activates controlled valve coupled to a respective solution inlet as well as the processor activates the micropump that is selectively coupled to a respective reservoir holding a respective solution, as provided in block 106. The micropump is activated for a prescribed amount of time that is associated with volume of interest for the solution, followed by deactivation of both the micropump and controlled valve, as collectively provided in block 108. Again, the present disclosure is not limited to two such controlled valves, solution inlets, or holding channels, rather a number greater than 2 can be realized depending on the need. If the answer to query 102 was “Y” (i.e., all the solutions have been loaded into the respective loading channels), then the processor proceeds to block 110 where the processor activates the controlled fluidic force inlet (i.e., opens the inlet). Next the processor proceeds to query 112, where the processor by receiving signals from an optional pressure sensor (block 114), and/or a mass air flow sensor (block 116), determines if sufficient fluidic force has been introduced into the controlled fluidic force inlet to cause the solutions in the holding channels to be moved to the mixing channel. If the answer to query 112 is “N” the processor activates the fluid force generator which can be a pump, a controlled compressed gas canister, etc., or the vacuum system as collectively shown in block 118. This loop between query 112 and block 118 continues until sufficient fluidic force has been introduced. If the answer to query 112 is “Y” (i.e., sufficient fluidic force has been introduced), the processor moves to block 120 and 121 where first the controlled fluid force inlet is deactivated (block 120) followed by deactivation of fluid force generator or deactivation of the vacuum system, collectively shown in block 121, which signifies successful mixing of all the solutions and the movement to the trap zone. Thereafter, the processor operates the microscope system (block 122) utilizing the optional temperature sensor (block 124), as discussed above.

While in certain embodiments described herein, the number of solution inlets and holding channels for introduction of solutions has been identified as two (e.g., a first and a second), as provided herein, this number is not limited to two and can be more, e.g., 3, 4, or even higher depending on the available physical size restriction of the microfluidic device. Thus, no limitation as to only two sets of such elements should be applied to the present disclosure.

Those having ordinary skill in the art will recognize that numerous modifications can be made to the specific implementations described above. The implementations should not be limited to the particular limitations described. Other implementations may be possible.

1. A microfluidic device, comprising:
two or more solution inlets each adapted to receive a respective solution, each of the two or more solution inlets adapted to be selectively opened and closed;
two or more loading channels each coupled to a respective solution inlet, each loading channel adapted to hold the respective solution;
a fluidic force inlet coupled to the two or more solution inlets and adapted to provide a fluidic force to each respective loading channel via the respective solution inlet; and
a mixing channel coupled to the two or more loading channels, thereby adapted to mix each solution held in each of the two or more loading channels when the fluidic force is applied to the fluidic force inlet, the mixing channel terminating at a trap zone adapted to receive the mixed solution from the mixing channel when exposed to the fluidic force, the trap zone including a fluidic force outlet adapted to release the received fluidic force.

2. The microfluidic device of claim 1, wherein one or more of the two or more loading channels is serpentine-shaped, thus adapted to hold a predetermined amount of the respective solution.

3. The microfluidic device of claim 1, wherein the mixing channel is serpentine shaped, thus adapted to provide sufficient volume for mixing of the respective solutions based on a predetermined volume criterion, wherein the mixing channel is defined by a length of between about 1 cm and about 20 cm and a diameter of about 1 μm and about 1 mm.

4. The microfluidic device of claim 1, wherein each of the two or more solution inlets adapted to be selectively closed and opened utilizing an inlet closing device.

5. The microfluidic device of claim 4, wherein the inlet closing device is a pressure sensitive adhesive.

6. The microfluidic device of claim 1, the fluidic force inlet is adapted to receive the fluid force from a syringe.

7. A microfluidic system, comprising:
a microfluidic device, including:
two or more solution inlets each adapted to receive a respective solution, each of
the two or more solution inlets adapted to be selectively opened and closed;
two or more loading channels each coupled to a respective solution inlet, each loading channel adapted to hold the respective solution;
a fluidic force inlet coupled to the two or more solution inlets and adapted to provide a fluidic force to each respective loading channel via the respective solution inlet; and
a mixing channel coupled to the two or more loading channels, thereby adapted to mix each solution held in each of the two or more loading channels when the fluidic force is applied to the fluidic force inlet, the mixing channel terminating at a trap zone adapted to receive the mixed solution from the mixing channel when exposed to the fluidic force, the trap zone including a fluidic force outlet adapted to release the received fluidic force;
a fluidic force device coupled to the fluidic force inlet adapted to generate the fluid force; and
a microscope system optically coupled to the trap zone adapted to irradiate the mixed solution with a light source and return optical emission from the mixed solution to an image capture device.

8. The microfluidic system of claim 7, wherein one or more of the two or more loading channels is serpentine-shaped, thus adapted to hold a predetermined amount of the respective solution.

9. The microfluidic system of claim 7, wherein the mixing channel is serpentine shaped, thus adapted to provide sufficient volume for mixing of the respective solution based on a predetermined volume criterion, wherein the mixing channel is defined by a length of between about 1 cm and about 20 cm and a diameter of about 1 μm and about 1 mm.

10. The microfluidic system of claim 7, wherein each of the two or more solution inlets adapted to be selectively closed and opened based on application of an inlet closing device.

11. The microfluidic system of claim 10, wherein the inlet closing device is a pressure sensitive adhesive.

12. The microfluidic system of claim 7, the fluidic force device is syringe.

13. The microfluidic system of claim 7, further comprising:
a pressure sensor coupled to the fluidic force inlet adapted to generate a signal associated with pressure in the two or more loading channels and the mixing channel.

14. The microfluidic system of claim 13, further comprising:
a processor executing software on a non-transient memory, configured to:
receive a pressure signal from the pressure sensor;
control the fluidic force device to thereby selectively apply a fluidic force based on a predetermined schedule in response to the received pressure signal.

15. The microfluidic system of claim 14, further comprising:
a temperature sensor disposed about the trap zone and adapted to provide a temperature signal associated with temperature of the mixed solution in the trap zone.

16. The microfluidic system of claim 15, wherein the processor is further configured to determine size of particles in the solution in the trap zone by application of particle diffusometry.

17. The microfluidic system of claim 16, wherein application of particle diffusometry includes steps of:
dividing a field of view of images captured by the image capture device into one or more segments;
for each of the one or more segments:
identify particles at two or more time intervals:
for each of the two or more time intervals:
obtain autocorrelation of first plurality of particles for a first timeslot according to locations of said first plurality of particles at the first timeslot,
identify an autocorrelation peak in the autocorrelation,
obtain cross-correlation of second plurality of particles for a second timeslot according to locations of said second plurality of particles as compared to said first plurality of particles from the first timeslot,
identify a cross-correlation peak in the cross-correlation,
for each identified autocorrelation and cross-correlation peaks, determine peak widths at 1/e of each peak, and
determine time-dependent particle size based on the determined peak widths as a function of time.

18. The microfluidic system of claim 17, wherein the time-dependent particle size is determined based on:
$${d}_{p} = \frac{16 {k}_{B} T Δ t {M}^{2}}{3 π ( {s}_{o , c}^{2} - {s}_{o , a}^{2} )} ,$$
wherein dₚ is time-dependent diameter of particles,
k_{B} is the Boltzmann constant,
T is temperature of the mixed solution,
μ is the mixed solution viscosity,
M is the magnification of the microscope of the microscope system, and
s_{o,c}² and s_{o,a}² are peak widths of peaks of the cross-correlation and autocorrelation, respectively.

19. The microfluidic system of claim 17, wherein the processor is further configured to determine time-dependent binding performance between particles suspended in the respective solutions of the two or more loading channels.

20. The microfluidic system of claim 17, wherein the time-dependent binding performance is expressed as k_{obs}t, expressed as:
$${k}_{obst} t = log ( \frac{{V}_{m} - V ( t )}{{V}_{m} - {V}_{0}} ) ,$$
wherein V₀ represents initial volume of particles in one of the respective solutions of the two or more loading channels, and
V(t) represents time-dependent volume of the particles in the mixed solution based on the time-dependent particle size.
